(19) United States
(12) Patent Application Publication
(10) Pub. No.: US 2013/0216617 A1
(43) Pub. Date: Aug. 22, 2013

(54) PHARMACEUTICAL COMPOSITIONS OF (R)-LANSOPRAZOLE

(71) Applicants: Sunilendu Bhushan Roy, Gujarat (IN); Sushrut Krishnaji Kulkarni, Gujarat (IN); Manohar Vishwanath Lalge, Gujarat (IN); Vaibhav Panditrao Deshmukh, Gujarat (IN)

(72) Inventors: Sunilendu Bhushan Roy, Gujarat (IN); Sushrut Krishnaji Kulkarni, Gujarat (IN); Manohar Vishwanath Lalge, Gujarat (IN); Vaibhav Panditrao Deshmukh, Gujarat (IN)

(73) Assignee: CADILA HEALTHCARE LIMITED, Ahmedabad (IN)

(21) Appl. No.: 13/807,439

(22) PCT Filed: Jun. 28, 2011
(86) PCT No.: PCT/IN2011/000429

§ 371 Date: Mar. 7, 2013

(30) Foreign Application Priority Data
Jun. 29, 2010 (IN) 1888/MUM/2010

(51) Int. Cl. A61K 9/50; A61K 9/20; A61K 31/4439

(52) U.S. Cl. CPC A61K 9/5073; A61K 31/4439; A61K 9/2081
USPC 424/452; 424/490; 514/338; 424/494; 424/497; 424/496; 424/465; 427/2.14

(57) ABSTRACT

The present invention relates to stable pharmaceutical compositions of (R)-lansoprazole or pharmaceutically acceptable salts thereof and process of preparing the same. The invention particularly provides pharmaceutical compositions of optically active (R)-isomer of lansoprazole with at least two functional coating layers.

### FIELD OF THE INVENTION

The present invention relates to stable pharmaceutical compositions of (R)-lansoprazole or pharmaceutically acceptable salts thereof. In particular, the invention provides pharmaceutical compositions of optically active (R)-isomer of lansoprazole with at least two functional coating layers and processes for preparing such compositions.

### BACKGROUND OF THE INVENTION

Substituted benzimidazoles (proton pump inhibitors) are potent inhibitors of gastric acid secretion. The susceptibility of these active proton pump inhibitor substances to degradation and transformation in acid media increases the difficulty of preparing a pharmaceutical form designed for oral administration. If the active substance comes in contact with the stomach content, which is a highly acidic medium, these chemical substances becomes more susceptible to acidic degradation. Thus, these benzimidazole derivatives should be protected both during storage and during their passage through the acidic environment of the stomach. Therefore, such labile drugs need to be formulated in a way to stabilize the compositions.

Lansoprazole is one of the most useful proton pump inhibitors useful in the treatment of disorders associated with gastric acid secretion. The optically active enantiomer of lansoprazole is (R)-lansoprazole, which is also known as dexlansoprazole. Dexlansoprazole is indicated for healing of all grades of erosive esophagitis and in the treatment of heartburn associated with non-erosive gastroesophageal reflux disease (GERD). Chemically, dexlansoprazole is (+)-2-[(R)-{([3-methyl-4-(2,2,2-trifluoroethoxy)pyridin-2-yl]methyl}sulfinyl]-1H-benzimidazole.

Presently, dexlansoprazole is administered as delayed release capsules. It is marketed in USA under the brand name Dexilant® by Takeda in the strengths of 30 and 60 mg.

U.S. Pat. No. 4,628,098 discloses racemic form of lansoprazole. U.S. Pat. Nos. 6,462,058; 6,664,276; 7,285,668; 7,339,064 and U.S. Application No. 2008/306118 disclose different crystalline forms of (R)-lansoprazole and its salts.

U.S. Application No. 2009/163553 discloses amorphous form of (R)-lansoprazole. U.S. Pat. No. 5,045,321 discloses a pharmaceutical composition in the form of coated tablets or granules, which is comprised of lansoprazole being in contact with at least one of the basic inorganic salts evenly.

U.S. Application No. 2003/008903 discloses a method of treating ulcers by administering a pharmaceutical composition comprising lansoprazole, containing at least 90% by weight (R)-lansoprazole and 10% or less by weight of (S)-lansoprazole, or a pharmaceutically acceptable salt thereof, and a pharmaceutically acceptable solid state carrier.

U.S. Pat. No. 7,790,755 discloses a controlled release preparation, in particular a capsule comprising a tablet, granule or fine granule wherein the release of active ingredient is controlled and a gel-forming polymer which delays the migration speed in the gastrointestinal tract.

U.S. Application No. 2005/003005 discloses stabilized granules, which comprises a high content (about 12% by weight or more based on the total granule weight) of an acid-unstable medicament, in particular, a benzimidazole compound useful as an antiulcer agent and a basic inorganic substance.

U.S. Application No. 2009/068263 discloses a multiple unit composition comprising of enteric coated pellets, each pellet comprises a core comprising active ingredient(s); optionally a separating layer coated on the core; and at least two enteric layers comprising of enteric polymers and plasticizer and the composition may avoid any change in the release profile of acid labile active ingredient on compression of enteric coated pellets into tablets and to ensure that the acid labile drug is prevented from being released in the gastric region.

U.S. Application No. 2007/196485 discloses a stable composition with a benzimidazole derivative, which does not contain a separating layer between the active compound and an enteric coating layer. Instead, the enteric coating layer is applied as a solution with a pH value of at least 6.5 directly to the benzimidazole derivative substrate.

U.S. Application No. 2009/208575 discloses a pharmaceutical composition for oral use comprising a) a core comprising an effective amount of benzimidazole and an organic stabilizing agent which is present in an amount effective to stabilize the composition, b) an intermediate layer comprising of a water insoluble polymer and an organic stabilizer, and c) an outer enteric coating layer.

Although several attempts have been made in the prior art to devise formulations of acid unstable benzimidazole derivatives (e.g. (R)-lansoprazole) with an objective to delay drug release or maintaining a consistent drug release profile and to provide a stable formulation, there still exists a continued need of an improved delayed release pharmaceutical compositions of (R)-lansoprazole or salt thereof, which not only provides drug release in the GIT at specified time interval and at specified release rate, but also simultaneously exhibiting excellent stability over the storage period as well as during their passage through the gastric (acidic) environment of the stomach.

### SUMMARY OF THE INVENTION

In one general aspect there is provided a stable pharmaceutical composition comprising a core comprising (R)-lansoprazole or pharmaceutically acceptable salts thereof and one or more pharmaceutically acceptable excipients; and at least two functional coating layers over the core, wherein the first functional coating layer comprises a polymeric substance which is soluble in the pH range of 6.0 to 7.5 and the second functional coating layer comprises a polymeric substance which is soluble in the pH range of 5.0 to 6.0.

In another general aspect there is provided a stable pharmaceutical composition comprising a dosage form coated with a functional coating layer comprising a polymeric substance, wherein the dosage form comprises:
(i) at least one core or pellet coated with a functional coating layer comprising (R)-lansoprazole or pharmaceutically acceptable salts thereof and one or more pharmaceutically acceptable excipients; and
(ii) a functional coating layer over the core or pellet comprising a polymeric substance, wherein the polymeric substance in the functional coating layer over the dosage form is different than the polymeric substance in the functional coating layer over the core or pellet.

Embodiments of the pharmaceutical composition may include one or more of the following features. For example, the pharmaceutical composition may further include one or more pharmaceutically acceptable excipients. The pharmaceutically acceptable excipients may include one or more binders, fillers, lubricants, disintegrants, stabilizers, glidants, and the like.

In another general aspect there is provided a stable capsule composition comprising multiparticulate units, each unit comprising a core comprising (R)-lansoprazole or pharmaceutically acceptable salts thereof and one or more pharmaceutically acceptable excipients; and at least two functional coating layers over the core, wherein the first functional coating layer comprises a polymeric substance which is soluble in the pH range of 6.0 to 7.5 and the second functional coating layer comprises a polymeric substance which is soluble in the pH range of 5.0 to 6.0.

Embodiments of the pharmaceutical composition may include one or more of the following features. For example, the pharmaceutical composition may further include one or more pharmaceutically acceptable excipients. The pharmaceutically acceptable excipients may include one or more binders, fillers, lubricants, disintegrants, stabilizers, glidants, and the like.

In another general aspect there is provided a stable pharmaceutical composition comprising:
(a) an inert core;
(b) at least one drug layer over the inert core comprising (R)-lansoprazole or pharmaceutically acceptable salts thereof and one or more pharmaceutically acceptable excipients;
(c) at least one intermediate layer over the drug layer; and
(d) at least two functional coating layers over the intermediate layer,
wherein the first functional coating layer comprises a polymeric substance which is soluble in the pH range of 6.0 to 7.5 and the second functional coating layer comprises a polymeric substance which is soluble in the pH range of 5.0 to 6.0.

In another general aspect there is provided a stable pharmaceutical composition comprising:
(a) a core comprising (R)-lansoprazole or pharmaceutically acceptable salts thereof and one or more pharmaceutically acceptable excipients;
(b) at least one intermediate layer over the drug layer;
(c) a first functional coating layer over the intermediate layer of step (b) comprising a polymeric substance which is soluble in the pH range of 6.0 to 7.5;
(d) at least one intermediate layer over the first functional coating layer;
(e) a drug layer over the intermediate layer of step (d) comprising (R)-lansoprazole or pharmaceutically acceptable salts thereof and one or more pharmaceutically acceptable excipients;
(f) at least one intermediate layer over the drug layer of step (e); and
(g) a second functional coating layer over the intermediate layer of step (f) comprising a polymeric substance which is soluble in the pH range of 5.0 to 6.0.

In another general aspect there is provided a stable pharmaceutical composition comprising a core comprising (R)-lansoprazole or pharmaceutically acceptable salts thereof, one or more stabilizers, one or more pharmaceutically acceptable excipients; and at least two functional coating layers over the core, wherein the first functional coating layer comprises a polymeric substance which is soluble in the pH range of 6.0 to 7.5 and the second functional coating layer comprises a polymeric substance which is soluble in the pH range of 5.0 to 6.0 and the stabilizer is present in an amount of at least 0.6 part by weight based on 1 part by weight of (R)-lansoprazole or pharmaceutically acceptable salts thereof.

Embodiments of the pharmaceutical composition may include one or more of the following features. For example, the polymeric substance of the first and second functional coating layer may be pre-neutralized to a suitable pH using alkaline substance. The drug core, drug layer, first functional coating layer and the second functional coating layers may be separated by one or more intermediate layers comprising one or more pharmaceutically acceptable excipients.

The pharmaceutical composition may further include one or more pharmaceutically acceptable excipients. The pharmaceutically acceptable excipients may include one or more binders, fillers, lubricants, disintegrants, stabilizers, glidants and the like.

In still another general aspect, there is provided a process for preparing a stable pharmaceutical composition, the process comprising:
(a) preparing a core comprising (R)-lansoprazole or pharmaceutically acceptable salts thereof and one or more pharmaceutically acceptable excipients;
(b) providing a first functional coating layer over the core comprising a polymeric substance which is soluble in the pH range of 6.0 to 7.5;
(c) providing a second functional coating layer over the core comprising a polymeric substance which is soluble in the pH range of 5.0 to 6.0; and
(d) formulating the core prepared in step (c) into a suitable dosage form.

In still another general aspect, there is provided a process for preparing a stable pharmaceutical composition, the process comprising:
(a) providing an inert core;
(b) providing at least one drug layer over the inert core comprising (R)-lansoprazole or pharmaceutically acceptable salts thereof and one or more pharmaceutically acceptable excipients;
(c) providing a first functional coating layer over the drug layer comprising a polymeric substance which is soluble in the pH range of 6.0 to 7.5;
(d) providing a second drug layer over the first functional coating layer comprising (R)-lansoprazole or pharmaceutically acceptable salts thereof and one or more pharmaceutically acceptable excipients;
(e) providing a second functional coating layer over the second drug layer comprising a polymeric substance which is soluble in the pH range of 5.0 to 6.0; and
(f) formulating the core prepared in step (e) into a suitable dosage form.

Embodiments of the pharmaceutical composition may include one or more of the following features. For example, the pharmaceutical composition may further include one or more pharmaceutically acceptable excipients. The pharmaceutically acceptable excipients may include one or more binders, fillers, lubricants, disintegrants, stabilizers, glidants, and the like.

In another general aspect, there is provided a stable pharmaceutical composition comprising a core comprising (R)-lansoprazole or pharmaceutically acceptable salts thereof and one or more pharmaceutically acceptable excipients; and at least two functional coating layers, wherein the first functional coating layer comprises a polymeric substance which is soluble in the pH range of 6.0 to 7.5 and the second functional coating layer comprises a polymeric substance which is soluble in the pH range of 5.0 to 6.0; wherein the composition retains at least 80% of potency of (R)-lansoprazole or pharmaceutically acceptable salts thereof after storage for three months at 40° C. and 75% relative humidity.

In another general aspect, there is provided a stable pharmaceutical composition comprising a core comprising (R)-lansoprazole or pharmaceutically acceptable salts thereof and one or more pharmaceutically acceptable excipients; and at least two functional coating layers, wherein the first functional coating layer comprises a polymeric substance which is soluble in the pH range of 6.0 to 7.5 and the second functional coating layer comprises a polymeric substance which is soluble in the pH range of 5.0 to 6.0 and said composition exhibits no significant difference in rate and/or extent of absorption of (R)-lansoprazole as compared to marketed formulation of (R)-lansoprazole available under the trade name Dexilant®.

Embodiments of the pharmaceutical composition may include one or more of the following features. For example, the pharmaceutical composition may further include one or more pharmaceutically acceptable excipients. The pharmaceutically acceptable excipients may include one or more binders, fillers, lubricants, disintegrants, stabilizers, glidants, and the like.

The details of one or more embodiments of then invention are set forth in the description below. Other features, objects and advantages of the invention will be apparent from the description.

### DETAILED DESCRIPTION OF THE INVENTION

The inventors of the present invention have devised a formulation of (R)-lansoprazole which demonstrates multiple release profile of the drug in the gastro-intestinal tract (GIT) at specified time interval and at specified release rate.

In particular, the inventors have also found that the formulation of the invention provides equivalent or relatively retarded drug release profile or exhibiting no significant difference when compared to release profile demonstrated by marketed formulation of (R)-lansoprazole available under the trade name Dexilant®.

Moreover, the formulations in accordance with the present invention also exhibit excellent storage stability, retaining at least 80% of the potency of (R)-lansoprazole or pharmaceutically acceptable salts thereof in the pharmaceutical composition after storage for three months at 40° C. and 75% relative humidity. Advantageously, the active ingredient, (R)-lansoprazole in composition is also protected during their passage through the gastric (acidic) environment of the stomach and thus ensures its delivery in therapeutically effective amount to lower parts of GIT.

The inventors have found that when more than one functional coating layers containing different polymeric substances (e.g. having different solubility over the gastrointestinal pH range (4.5 to 7.5)), the resulting composition demonstrates a multiple release profile of (R)-lansoprazole over a specified time interval and with a specific release rate. Thus, the formulations of the present invention thwart the need of providing separate dosage units where each dosage unit provides a separate drug release profile.

Thus, the embodiments of the present invention relates to the formulation of (R)-lansoprazole comprising a core comprising (R)-lansoprazole or pharmaceutically acceptable salts thereof and one or more pharmaceutically acceptable excipients; and at least two functional coating layers, wherein the polymeric substances in both the functional coatings are different. In particular, the first functional coating layer may include a polymeric substance which is soluble in the pH range of 6.0 to 7.5 and the second functional coating layer may include a polymeric substance which is soluble in the pH range of 5.0 to 6.0.

The term ‘(R)-lansoprazole’ used throughout the specification refers to (R)-lansoprazole per se, its pharmaceutically acceptable salts, pharmaceutically acceptable solvates, pharmaceutically acceptable hydrates, pharmaceutically acceptable derivatives, pharmaceutically acceptable polymorphs and pharmaceutically acceptable prodrugs thereof, its various crystalline or amorphous forms or mixtures thereof, and its mixture with other enantiomer ((S)-lansoprazole).

The term “core” as described herein refers to a tablet or multiparticulates such as granules, pellets, beads, spheres, minitablets or microtablets. The core may be prepared by known techniques such as direct compression, wet granulation, dry granulation, melt granulation or extrusion-spheronization, and spray coating or layering.

The “inert core” may comprise inert non-pareils which are conventionally used in pharmaceutical industry and are readily available. The inert non-pareils may be of any pharmaceutically acceptable excipient such as starch, sugar, microcrystalline cellulose, vegetable gums, waxes, and the like. Preferably, the inert non-pareils are of starch and sugar. The size of the inert non-pareils may vary from 0.1 mm-2 mm.

When an inert core is used, non-pareils may be coated with a seal coat comprising a film forming polymer, e.g. ethylcellulose, and excipients like plasticizer, anti-tacking agent and opacifying agent. The components of the seal coat may be dissolved or dispersed in an appropriate solvent and the dispersion may be coated on the core in a conventional coating pan or fluidized bed equipment (such as a Wurster or Glatt) and the coated cores may then be dried. A coat of (R)-lansoprazole may then be applied to such coated cores using similar process as above, wherein (R)-lansoprazole may be built up on the coated cores by spraying a suspension or dispersion comprising (R)-lansoprazole and excipients such as binder, plasticizer, anti-tacking agent and opacifying agent. Alternatively, the (R)-lansoprazole coat may also be applied by powder-coating, wherein the coated cores as described above are maintained in a sticky state, a mixture of (R)-lansoprazole and powdered excipients such as binder, plasticizer, anti-tacking agent and opacifying agent are added continuously or periodically so as to adhere to the sticky cores. When the entire (R)-lansoprazole coat has been applied, the drug coated cores may be dried.

The active ingredient-containing core thus obtained may be further coated to provide an intermediate layer. The “intermediate layer” may provide stability by inhibiting direct contact of the components of the core and the polymeric substance in the functional coating layer. The intermediate layer may also provide protection to the core during its passage from the stomach to the intestines. Preferably, the separating coat is compatible with (R)-lansoprazole or salts thereof and the functional layer and does not affect the dissolution of the composition. The intermediate layer may be prepared by dissolving an appropriate amount of film forming polymer into a suitable solvent system such as water, organic solvent such as alcohol, methylene chloride, and the like; or mixtures thereof, and spraying the solution or suspension on core or suitable layer using a suitable apparatus. The intermediate coating layer may be formed by a plural number of layers. The intermediate layer may comprise a stabilizer.

The coating materials for the intermediate layer may include low substituted hydroxypropyl cellulose, hydroxypropyl cellulose, hydroxypropyl methylcellulose, polyvinylpyrrolidone, polyvinyl alcohol, methylcellulose and hydroxyethyl methylcellulose. Excipients (for example, masking agents (titanium oxide and the like) and antistatic agents (titanium oxide, talc and the like) may be suitably added to the intermediate layer, if necessary.

The drug-containing cores or intermediate layer may be coated with at least two functional coating layers. The polymeric substance used in functional coating layers may include polymers such as hydroxypropyl methylcellulose phthalate, cellulose acetate phthalate, cellulose acetate succinate, methylcellulose phthalate, hydroxypropyl methylcellulose phthalate, ethylhydroxycellulose phthalate, polyvinylacetate phthalate, polyvinyl butyrate acetate, vinyl acetate-maleic anhydride copolymer, styrene-maleic mono-ester copolymer, carboxymethyl ethylcellulose, methyl methacrylate-methacrylic acid copolymer (Eudragit L-100 (methacrylic acid copolymer L) or Eudragit S-100 (methacrylic acid copolymer S)), methacrylic acid-ethyl acrylate copolymer (Eudragit L100-55 (dried methacrylic acid copolymer LD) or Eudragit L30D-55 (methacrylic acid copolymer LD)), methacrylic acid-methyl acrylate-methyl methacrylate copolymer (Eudragit FS30D), hydroxypropyl cellulose acetate succinate (HPMCAS), shellac or combinations thereof. The polymeric substance as the above-mentioned coating material may be used alone or two or more kinds of the polymers may be used to coat in combination. The first functional coating layer may include a polymeric substance such that it is soluble in the pH range of 6.0 to 7.5 and the second functional coating layer may include a polymeric substance such that it is soluble in the pH range of 5.0 to 6.0.

The polymeric substance, such as methacrylic acid copolymer, may be neutralized to an appropriate pH by using alkaline substances, such as sodium hydroxide, potassium hydroxide or ammonium hydroxide; and the like, and the neutralized polymer may be used in preparation of the functional coating layer. The functional coating layer may be applied by dispersing or suspending the polymeric substance in a suitable medium, such as water or aqueous acidic or alkaline solutions, or in organic solvents such as methanol, ethanol, isopropanol, acetone, methyl ethyl ketone, methylene chloride, ethylene chloride, ethyl acetate, or mixtures thereof, and the resultant solution or suspension may be sprayed directly on the core or intermediate layer, followed by drying to obtain the stable pharmaceutical composition.

Suitable dosage form may include one or more of tablets, multilayered tablets, capsules, pellets, granules, spheroids, beads, minitablets in capsule, pellets (multiparticulate units) in capsule, granules in capsule, powder. Further the powder or granules can be suspended to give a pharmaceutically acceptable oral suspension.

In an embodiment, the pharmaceutical composition may include a capsule coated with a functional coating layer comprising a polymeric substance which is soluble in the pH range of 5.0 to 6.0, which capsule further comprises multiple cores (e.g. tablets or mini-tablets) or pellets comprising (R)-lansoprazole or pharmaceutically acceptable salts thereof and one or more pharmaceutically acceptable excipients, wherein said multiple cores (e.g. tablets or mini-tablets) or pellets are coated with a functional coating layer comprising a polymeric substance which is soluble in the pH range of 6.0 to 7.5.

The pharmaceutically acceptable excipients may include one or more fillers, disintegrants, binders, stabilizers, lubricants, glidants, and the like.

Suitable fillers may include one or more of microcrystalline cellulose, starch, dibasic calcium phosphate, tribasic calcium phosphate, calcium carbonate, dextrose, kaolin, magnesium carbonate, magnesium oxide; sugars such as lactose or sucrose; sugar alcohols such as mannitol, sorbitol, erythritol, and the like.

Suitable disintegrants may include one or more of croscarmellose sodium, sodium starch glycolate, pregelatinized starch, sodium carboxymethyl cellulose, cross-linked polyvinylpyrrolidone, and the like.

Suitable binders may include one or more of hydroxyethyl cellulose, hydroxypropyl cellulose, hydroxypropyl methylcellulose, carbomers, dextrin, ethyl cellulose, methylcellulose, shellac, zein, gelatin, polymethacrylates, polyvinyl pyrrolidone, pregelatinized starch, sodium alginate, gums, synthetic resins, and the like.

The pharmaceutical composition of the present invention preferably contain one or more stabilizers such as basic inorganic salts, capable of imparting stability to the active agent present in the composition.

Suitable stabilizers may include a basic inorganic stabilizer or an organic stabilizer or combination thereof. Suitable basic inorganic stabilizer may include one or more of magnesium carbonate, magnesium oxide, magnesium hydroxide, magnesium aluminate metasilicate, magnesium silicate, magnesium aluminate, synthetic hydrotalcite, alumina magnesium hydroxide, calcium carbonate, calcium hydroxide, sodium carbonate, sodium bicarbonate, sodium hydroxide, potassium carbonate, potassium bicarbonate, potassium hydroxide and the like. Suitable organic stabilizer may include one or more of meglumine, triethanolamine, tromethamine, tris(hydroxymethyl)aminomethane (TRIS), monosodium glutamate, polacrillin sodium, sodium alginate and the like. The stabilizer present in the core or drug layer is in an amount of at least 6%, preferably at least 7% by weight based on 1 part by weight of (R)-lansoprazole or salts thereof. In a particularly preferred embodiment, the stabilizer in the composition is preferably present in an amount of at least 0.6 parts by weight based on 1 part by weight of (R)-lansoprazole or pharmaceutically acceptable salts thereof.

Suitable lubricants and glidants may include one or more of talc, metallic stearates such as magnesium stearate, calcium stearate, zinc stearate; colloidal silicon dioxide, finely divided silicon dioxide, stearic acid,.hydrogenated vegetable oil, glyceryl palmitostearate, glyceryl monostearate, glyceryl behenate, polyethylene glycols, powdered cellulose, starch, sodium stearyl fumarate, sodium benzoate, mineral oil, magnesium trisilicate, kaolin; and the like

The pharmaceutical compositions as described herein may be prepared by processes known to a person having ordinary skill in the art of pharmaceutical technology such as direct compression, wet granulation, dry granulation, fluidized bed granulation, melt granulation, melt extrusion, spray coating, spray drying and solution evaporation.

The inventors have further surprisingly found that when drug cores and drug layers are prepared by wet/ dry granulation method and aqueous based (using water as vehicle) respectively, resulting formulations possess improved stability,

In an embodiment, the process of preparing a stable pharmaceutical composition of (R)-lansoprazole or pharmaceutically acceptable salts thereof comprises the steps of:
(a) preparing a core comprising (R)-lansoprazole or pharmaceutically acceptable salts thereof and one or more pharmaceutically acceptable excipients;
(b) providing a first functional coating layer over the core comprising a polymeric substance which is soluble in the pH range of 6.0 to 7.5;
(c) providing a second functional coating layer over the core comprising a polymeric substance which is soluble in the pH range of 5.0 to 6.0; and
(d) formulating the core prepared in step (c) into a suitable dosage form.

In a further embodiment, the stable pharmaceutical composition of (R)-lansoprazole or pharmaceutically acceptable salts thereof may be prepared by:
(a) preparing a core by granulating and/or mixing & compressing (R)-lansoprazole or pharmaceutically acceptable salts thereof with a stabilizer and one or more pharmaceutically acceptable carrier;
(b) applying a first functional coating layer comprising methacrylic acid copolymer which is soluble in the pH range of 6.0 to 7.5 over the core; and
(b) applying a second functional coating layer comprising methacrylic acid copolymer which is soluble in the pH range of 5.0 to 6.0 over the core.

In a further embodiment, the process of preparing a stable pharmaceutical composition of (R)-lansoprazole or pharmaceutically acceptable salts thereof comprises the steps of:
(a) providing an inert core;
(b) providing at least one drug layer over the inert core comprising (R)-lansoprazole or pharmaceutically acceptable salts thereof and one or more pharmaceutically acceptable excipients;
(c) providing a first functional coating layer over the drug layer comprising a polymeric substance which is soluble in the pH range of 6.0 to 7.5;
(d) providing a second drug layer over the first functional coating layer comprising (R)-lansoprazole or pharmaceutically acceptable salts thereof and one or more pharmaceutically acceptable excipients;
(e) providing a second functional coating layer over the second drug layer comprises a polymeric substance which is soluble in the pH range of 5.0 to 6.0; and
(f) formulating the cores prepared in step (e) into suitable dosage form.

In a further embodiment, the stable pharmaceutical composition of (R)-lansoprazole or pharmaceutically acceptable salts thereof may be prepared by:
(a) providing a core tablet or mini-tablet by mixing and granulating (R)-lansoprazole or pharmaceutically acceptable salts thereof with one or more pharmaceutically acceptable excipients; compressing the granules into a tablet;
(b) applying an intermediate coating layer over the core tablet/mini-tablet;
(c) applying a first functional coating layer over the intermediate coating layer comprising a polymeric substance which is soluble in the pH range of 6.0 to 7.5;
(d) applying an intermediate coating layer over the first functional coating layer;
(e) applying a drug layer over the intermediate coating layer of step (d) comprising (R)-lansoprazole or pharmaceutically acceptable salts thereof;
(f) applying an intermediate coating layer over the drug layer of step (e);
(g) applying a second functional coating layer over the intermediate coating layer of step (f) comprises a polymeric substance which is soluble in the pH range of 5.0 to 6.0.

The invention is further illustrated by the following examples which are provided to be exemplary of the invention and do not limit the scope of the invention. While the present invention has been described in terms of its specific embodiments, certain modifications and equivalents will be apparent to those skilled in the art and are intended to be included within the scope of the present invention.

### EXAMPLE 1

TABLE 1

| INGREDIENTS | Qty/unit (mg) |
|---|---|
| Drug layer - 1 | |
| Sugar spheres (Non-pareil seeds) | 40-60 |
| (R)-lansoprazole | 45 |
| Hydroxypropylcellulose | 6-15 |
| Sucrose | 8-18 |
| Magnesium carbonate/Meglumine/ | 4-40 |
| Purified water | q.s. |
| Intermediate layer | |
| Hypromellose | 6-16 |
| Talc | 2-8 |
| Purified water | q.s. |
| Functional coating layer - 1 | |
| Eudragit L-100 | 4-20 |
| Eudragit S-100 | 2-14 |
| Triethyl citrate/Polyethylene glycol | 2-10 |
| Titanium dioxide | 0-2 |
| Talc | 0-6 |
| Isopropyl alcohol | q.s. |
| Purified water | q.s. |
| Intermediate layer | |
| Hypromellose | 6-16 |
| Talc | 2-10 |
| Purified water | q.s. |
| Drug layer - 2 | |
| (R)-lansoprazole | 15 |
| Hydroxypropylcellulose | 0-10 |
| Sucrose | 0-6 |
| Magnesium carbonate/Meglumine/ | 1-14 |
| Purified water | q.s. |
| Intermediate layer | |
| Hypromellose | 6-26 |
| Talc | 2-14 |
| Purified water | q.s. |
| Functional coating layer - 2 | |
| Eudragit L30 D55 | 6-46 |
| Talc | 4-20 |
| Polyethylene glycol/Triethyl citrate | 0-6 |
| Titanium dioxide | 0-2 |
| Polysorbate | 0-4 |
| Purified water | q.s. |
| Lubrication | |
| Talc | 0-2 |
| Colloidal silicon dioxide | 0-2 |

### Process:

### A) Drug Layer—1:

Sucrose and hydroxypropylcellulose were dissolved in purified water. Magnesium carbonate/ Meglumine/ Triethanolamine was dispersed in purified water and milled. This milled dispersion was added into solution of sucrose with stirring. To this, (R)-lansoprazole was slowly added and dispersed to get drug coating suspension. This suspension was sprayed on sugar spheres by known technique to obtain drug loaded pellets.

### B) Intermediate Layer:

Hypromellose was dissolved in water and talc was dispersed in it to make suspension. This suspension was sprayed on drug loaded pellets to obtain barrier coated pellets.

### C) Functional Coating Layer—1:

Coating suspension was prepared by dispersing Eudragit L-100 & Eudragit S-100 in Isopropyl Alcohol, mixing with aqueous dispersion of titanium dioxide and talc; and dispersing triethyl citrate / polyethylene glycol. This suspension was sprayed on barrier coated pellets to obtain enteric coated pellets.

### D) Drug Layer—2

Enteric coated pellets were again coated with a barrier layer and then with a drug layer as prepared earlier with the specified quantity of ingredients.

### E) Functional Coating Layer—2:

Drug layered pellets of earlier step were again coated with a barrier layer.

Coating suspension for functional coating layer was prepared by dissolving polysorbate and polyethylene glycol / triethyl citrate in purified water, dispersing talc and titanium dioxide in it and then by dispersing Eudragit L30 D 55. This suspension was sprayed on barrier coated pellets to obtain enteric coated pellets.

### ) Lubrication:

The enteric coated pellets were lubricated with talc and colloidal silicon dioxide and filled into suitable size hard gelatin capsules.

### EXAMPLE 2

TABLE 2

| INGREDIENTS | Qty./unit |
|---|---|
| Core Tablet/Mini-Tablet | |
| (R)-lansoprazole | 45 |
| Hydroxypropylcellulose | 6-16 |
| Sucrose | 8-18 |
| Magnesium carbonate | 32-40 |
| Talc/Magnesium stearate | 0.5-4 |
| Intermediate layer | |
| Hypromellose | 1-10 |
| Talc | 0.5-6 |
| Purified water | q.s. |
| Functional coating layer - 1 | |
| Eudragit L-100 | 1-10 |
| Eudragit S-100 | 1-8 |
| Triethyl citrate/Polyethylene glycol | 1-8 |
| Titanium dioxide | 0-2 |
| Talc | 0.5-4 |
| Isopropyl alcohol | q.s. |
| Purified water | q.s. |
| Intermediate layer | |
| Hypromellose | 1-8 |
| Talc | 0.5-6 |
| Purified water | q.s. |
| Drug layer | |
| (R)-lansoprazole | 15 |
| Hydroxypropylcellulose | 1-10 |
| Sucrose | 1-6 |
| Magnesium carbonate | 12-14 |
| Purified water | q.s. |
| Intermediate layer | |
| Hypromellose | 1-8 |
| Talc | 0.5-6 |
| Purified water | q.s. |
| Functional coating layer - 2 | |
| Eudragit L30 D55 | 4-20 |
| Talc | 0.5-10 |
| Polyethylene glycol/Triethyl citrate | 1-4 |
| Titanium dioxide | 0-2 |
| Polysorbate | 0.5-4 |
| Purified water | q.s. |
| Lubrication (in case of Mini-tablet) | |
| Talc | 0-2 |
| Colloidal silicon dioxide | 0-2 |

### Process:

### A) Core Tablet/Mini-Tablet:

(R)-lansoprazole, hydroxypropylcellulose, sucrose and magnesium carbonate were mixed together and lubricated with talc/magnesium stearate. The lubricated blend was compressed to obtain a core tablet/mini-tablets.

### B) Intermediate Layer:

Hypromellose was dissolved in water and talc was dispersed in it to make suspension. This suspension was sprayed on core tablet/mini-tablets to obtain barrier coated tablet/mini-tablets.

### C) Functional Coating Layer—1:

Coating suspension was prepared by dispersing Eudragit L-100 & Eudragit S-100 in Isopropyl alcohol, mixing with aqueous dispersion of titanium dioxide and talc; and dispersing triethyl citrate/polyethylene glycol. This suspension was sprayed on barrier coated tablet/mini-tablets to obtain enteric coated tablet/mini-tablets.

### D) Drug Layer

Enteric coated tablet/mini-tablets were again coated with a barrier layer. Sucrose and hydroxypropylcellulose were dissolved in purified water. Magnesium carbonate was dispersed in purified water and milled. This milled dispersion was added into solution of sucrose with stirring. To this, (R)-lansoprazole was slowly added and dispersed to get drug coating suspension. This suspension was sprayed on barrier layered tablet/mini-tablets.

### E) Functional Coating Layer—2:

Drug layered tablet/mini-tablets were again coated with a barrier layer. Coating suspension for functional coating layer was prepared by dissolving polysorbate and polyethylene glycol/triethyl citrate in purified water, dispersing talc and titanium dioxide in it and then by dispersing Eudragit L30 D 55. This suspension was sprayed on barrier coated tablet/mini-tablets to obtain final tablet composition or enteric coated mini-tablets.

### F) Lubrication:

The enteric coated mini-tablets were lubricated with talc and colloidal silicon dioxide and filled into suitable size hard gelatin capsules.

### EXAMPLE 3

### Dissolution Study

TABLE 3

| | | Composition of the |
|---|---|---|
| | Dexilant ® | Invention |
| Time(min) | (% Drug release) | (% Drug release) |
| Acid Stage (2 hours) | 4.2 | 3.5 |
| 10 | 22.0 | 6.2 |
| 20 | 31.9 | 15.4 |
| 40 | 70.6 | 38.1 |
| 50 | 80.1 | 49.5 |
| 60 | 87.7 | 58.7 |
| 75 | 90.1 | 68.9 |
| 105 | 89.7 | 77.9 |
| 120 | 92.0 | 79.5 |

Table 3 provides dissolution data for reference formulation (Dexilant®) and the composition according to the invention. For determination of drug release rate, USP Type I apparatus (100 rpm) was used wherein 0.1N HCl (500 ml) was used for acid stage and Phosphate buffer (900 ml) of pH 7.0 with 5 mM of Sodium lauryl sulphate was used as intestinal stage medium. The results indicate that composition of the invention migrates the drug release in equivalent manner or even further relative to that exhibited by reference formulation.

### EXAMPLE 4

### Stability Study on Composition of the Invention Involving Drug Layering

TABLE 4

| | Aqueous Drug loading | | | | | Hydro-alcoholic drug loading | | | | |
|---|---|---|---|---|---|---|---|---|---|---|
| | Initial | 6 hr | 12 hr | 22 hr | 36 hr | Initial | 6 hr | 12 hr | 22 hr | 36 hr |
| Imp A | 0.07 | 0.08 | 0.08 | 0.11 | 0.11 | 0.08 | 0.08 | 0.09 | 0.11 | 0.12 |
| Imp C | 0.14 | 0.18 | 0.18 | 0.20 | 0.23 | 0.18 | 0.19 | 0.20 | 0.23 | 0.27 |
| Max | 0.06 | 0.08 | 0.08 | 0.08 | 0.10 | 0.08 | 0.11 | 0.12 | 0.21 | 0.29 |
| Unknown Imp | | | | | | | | | | |
| Total | 0.27 | 0.34 | 0.34 | 0.38 | 0.69 | 0.34 | 0.38 | 0.54 | 1.00 | 1.50 |

Table 4 provides stability data of the composition of the invention involving drug layering process. The result indicates that aqueous based drug loading process generates substantially less impurities that hydro-alcoholic drug loading process. Hence it is postulated that aqueous based drug loading process results in stable formulation.

### EXAMPLE 5

### Stability Study on Composition of the Invention in the Form of Drug Cores/Mini-Tablets

TABLE 5

| | Drug cores/Mini-tablets | Drug cores/Mini-tablets |
|---|---|---|
| | prepared by wet | prepared by dry |
| | granulation | granulation |
| Imp A | 0.16 | 0.14 |
| Imp C | 0.42 | 0.25 |
| Max Unknown Imp | 0.36 | 0.13 |
| Total | 2.30 | 1.10 |

Table 5 provides stability data of the composition of the invention in the form of drug cores/ mini-tablets. Taking into consideration the level of impurities, dry granulation process is more preferred over the wet granulation process in order to prepare stable composition of (R)-lansoprazole.

While the invention has been described in terms of its specific embodiments, certain modifications and equivalents will be apparent to those skilled in the art and are intended to be included within the scope of the invention.

We claim:

1. A stable pharmaceutical composition comprising a core comprising (R)-lansoprazole or pharmaceutically acceptable salts thereof and one or more pharmaceutically acceptable excipients; and at least two functional coating layers over the core, wherein the first functional coating layer comprises a polymeric substance which is soluble in the pH range of 6.0 to 7.5 and the second functional coating layer comprises a polymeric substance which is soluble in the pH range of 5.0 to 6.0.

2. The stable pharmaceutical composition as claimed in claim 1, wherein the core comprises an inert core coated with (R)-lansoprazole or pharmaceutically acceptable salts thereof and one or more pharmaceutically acceptable excipients.

3. The stable pharmaceutical composition as claimed in claim 1, wherein the composition further comprises at least one drug layer comprising (R)-lansoprazole or pharmaceutically acceptable salts thereof and one or more pharmaceutically acceptable excipients.

4. The stable pharmaceutical composition as claimed in claim 3, wherein the drug layer is formed over the first functional coating layer.

5. The stable pharmaceutical composition as claimed in claim 1, wherein the polymeric substance comprises one or more of hydroxypropylmethyl cellulose phthalate, cellulose acetate phthalate, carboxymethylethyl cellulose, methyl methacrylate-methacrylic acid copolymer, methacrylic acid-ethyl acrylate copolymer, methacrylic acid-methyl acrylate-methyl methacrylate copolymer, hydroxypropyl cellulose acetate succinate, polyvinyl acetate phthalate and shellac.

6. The stable pharmaceutical composition as claimed in claim 1, wherein the core further comprises one or more stabilizers.

7. The stable pharmaceutical composition as claimed in claim 6, wherein the stabilizer comprises one or more basic inorganic salts.

8. The stable pharmaceutical composition as claimed in claim 7, wherein the basic inorganic salt comprises one or more of sodium aluminium silicate, calcium silicate, magnesium aluminometasilicate, magnesium aluminosilicate, magnesium aluminum silicate, magnesium aluminate, dry aluminum hydroxide, synthetic hydrotalcite, synthetic aluminum silicate, magnesium carbonate, calcium carbonate, magnesium oxide, aluminum hydroxide, sodium hydrogencarbonate, sodium phosphate, disodium hydrogenphosphate, sodium dihydrogenphosphate, potassium phosphate, dipotassium hydrogenphosphate, potassium dihydrogenphosphate, disodium citrate, ammonium chloride, sodium carbonate, potassium carbonate, sodium bicarbonate, potassium hydrogen carbonate, sodium phosphate, potassium phosphate, calcium dibasic phosphate, trisodium phosphate, sodium hydroxide, potassium hydroxide and lithium hydroxide.

9. The stable pharmaceutical composition as claimed in claim 8, wherein the stabilizer is present in an amount of at least 0.6 parts by weight based on 1 part by weight of (R)-lansoprazole or pharmaceutically acceptable salts thereof.

10. The stable pharmaceutical composition as claimed in claim 1, wherein the composition is provided in the form of a tablet, a capsule, granules, pellets, caplets, minitablets, a capsule filled with minitablets and/or pellets, a multi-layer tablet, granules for suspension, or granules and powder filled in a sachet.

11. A stable pharmaceutical composition comprising a dosage form coated with a functional coating layer comprising a polymeric substance, wherein the dosage form comprises:
(i) at least one core or pellet coated with a functional coating layer comprising (R)-lansoprazole or pharmaceutically acceptable salts thereof and one or more pharmaceutically acceptable excipients; and
(ii) a functional coating layer over the core or pellet comprising a polymeric substance, wherein the polymeric substance in the functional coating layer over the dosage form is different than the polymeric substance in the functional coating layer over the core or pellet.

12. The stable pharmaceutical composition as claimed in claim 11, wherein the functional coating layer over the dosage form comprises a polymeric substance which is soluble in the pH range of 5.0 to 6.0.

13. The stable pharmaceutical composition as claimed in claim 11, wherein the functional coating layer over the core or pellet comprises a polymeric substance which is soluble in the pH range of 6.0 to 7.5.

14. The stable pharmaceutical composition as claimed in claim 11, wherein the dosage form is provided in the form of a tablet, capsule, caplets, minitablets, or a multi-layer tablet.

15. A stable pharmaceutical composition comprising:
(a) a core comprising (R)-lansoprazole or pharmaceutically acceptable salts thereof and one or more pharmaceutically acceptable excipients;
(b) at least one intermediate layer over the drug layer;
(c) a first functional coating layer over the intermediate layer of step (b) comprising a polymeric substance which is soluble in the pH range of 6.0 to 7.5;
(d) at least one intermediate layer over the first functional coating layer;
(e) a drug layer over the intermediate layer of step (d) comprising (R)-lansoprazole or pharmaceutically acceptable salts thereof and one or more pharmaceutically acceptable excipients;
(f) at least one intermediate layer over the drug layer of step (e); and
(g) a second functional coating layer over the intermediate layer of step (f) comprising a polymeric substance which is soluble in the pH range of 5.0 to 6.0.

16. A stable pharmaceutical composition as claimed in claim 1, wherein the composition exhibits no significant difference in rate and/or extent of absorption of (R)-lansoprazole as compared to marketed formulation of (R)-lansoprazole available under the trade name Dexilant®.

17. A stable pharmaceutical composition as claimed in claim 1, wherein the composition retains at least 80% of the potency of (R)-lansoprazole or pharmaceutically acceptable salts thereof in the pharmaceutical composition after storage for three months at 40° C. and 75% relative humidity.

18. A process for the preparation of a stable pharmaceutical composition, the process comprising the steps of:
(a) providing an inert core;
(b) providing at least one drug layer over the inert core comprising (R)-lansoprazole or pharmaceutically acceptable salts thereof and one or more pharmaceutically acceptable excipients;
(c) providing a first functional coating layer over the drug layer comprising a polymeric substance which is soluble in the pH range of 6.0 to 7.5;
(d) providing a second drug layer over the first functional coating layer comprising (R)-lansoprazole or pharmaceutically acceptable salts thereof and one or more pharmaceutically acceptable excipients;
(e) providing a second functional coating layer over the second drug layer comprising a polymeric substance which is soluble in the pH range of 5.0 to 6.0; and
(f) formulating the core prepared in step (e) into a suitable dosage form.

19. The process as claimed in claim 18, wherein the drug layering steps are carried out using aqueous based coating.
